(19) United States
(12) Patent Application Publication
(10) Pub. No.: US 2014/0051737 A1
(43) Pub. Date: Feb. 20, 2014

(54) METHODS FOR THE TREATMENT AND DIAGNOSTIC OF PULMONARY ARTERIAL HYPERTENSION

(71) Applicants: Sebastien Bonnet, Quebec (CA); Jean-Yves Masson, Quebec (CA); Guy Poirier, Quebec (CA)

(72) Inventors: Sebastien Bonnet, Quebec (CA); Jean-Yves Masson, Quebec (CA); Guy Poirier, Quebec (CA)

(73) Assignee: UNIVERSITE LAVAL, Quebec (CA)

(21) Appl. No.: 14/113,517

(22) PCT Filed: May 10, 2012
(86) PCT No.: PCT/CA2012/050306

§ 371 Date: Oct. 23, 2013

Related U.S. Application Data
(60) Provisional application No. 61/484,463, filed on May 10, 2011.

(51) Int. Cl. A61K 31/4184; C12Q 1/68; G01N 33/53; A61K 45/06

(52) U.S. Cl. CPC A61K 31/4184; A61K 45/06; C12Q 1/6876; G01N 33/53
USPC 514/394; 548/306.1; 435/6.12; 435/7.1

(57) ABSTRACT

The present application relates to the use at least one PARP inhibitor or a pharmaceutically acceptable salt thereof for the treatment of Group 1 pulmonary arterial hypertension (PAH) in a subject, including a human, in need of such treatment. There is also provided methods of treating and diagnosing Group 1 pulmonary arterial hypertension (PAH).

The present invention relates to methods for the treatment and diagnostic of pulmonary arterial hypertension.

Pulmonary arterial hypertension (PAH) is a vascular disease that is largely restricted to small pulmonary arteries. PAH occurs in rare idiopathic and familial forms, but is more commonly part of a syndrome associated with connective tissue diseases, anorexigen use, HIV or congenital heart disease. PAH, a multifactorial disease, is characterized by obstructed, constricted small pulmonary arteries (PA). This includes abnormalities in the blood content of some neurotransmitters and cytokines, namely increases in serotonin, IL-6, PDGF and endothelin. The media is also characterized by an increased activation of the nuclear factor of activated T-cells (NFAT) leading to increased [Ca2+]i-mediated PASMC proliferation, and decreased mitochondrial-dependent apoptosis (Bonnet et al., 2007a; Bonnet et al., 2006). Finally, the adventitia is infiltrated with inflammatory cells and exhibits metalloprotease activation (Humbert et al., 2004). Despite recent therapeutic advances such as endothelin-1 receptor blockers (e.g. bosentan) (Dupuis and Hoeper, 2008), type 5 phosphodiesterase inhibitors (e.g. sildenafil) (Li et al., 2007) or PDGF receptor blockers (e.g. imanitib) (Ghofrani et al., 2005), mortality rates remain high (Archer and Rich, 2000).

Poly(ADP-ribose) polymerases are defined as cell signalling enzymes that catalyze the transfer of ADP-ribose units from NAD⁺to a number of acceptor proteins. PARP-1, the best-characterized member of the PARP family, which currently comprises 18 members, is an abundant nuclear enzyme implicated in cellular responses to DNA injury provoked by genotoxic stress. PARP is involved in DNA repair and transcriptional regulation and is now recognized as a key regulator of cell survival and cell death as well as a master component of a number of transcription factors involved in tumour development and inflammation including NFAT. PARP becomes activated in response to oxidative DNA damage and depletes cellular energy pools, thus leading to cellular dysfunction in various tissues.

PARP inhibitors are currently being developed for the treatment of cancer. The inhibition of PARP is relevant for the treatment of cancers with specific DNA-repair defects, including those arising in carriers of a BRCA1 or BRCA2 mutation. Fong et al. (Fong et al., 2009) reported that the PARP inhibitor olaparib only showed objective antitumor activity in patients carrying the BRCA1 or BRCA2 mutation. PARP inhibitors therefore appear to be relevant for BRCA deficient cells.

PARP inhibition has been studied for the prevention of restenosis after endarterectomy (Beller et al., 2006). Abdallah et al. (2007) have showed that PARP inhibition can decrease endothelial cell proliferation. There is no evidence that PARP(s) is involved in pulmonary arterial hypertension or that PARP(s) expression level is modified in pulmonary arterial hypertension.

There is therefore a need for new methods for the treatment of PAH. There is also need for new methods for the diagnostic of PAH.

One embodiment of the invention relates to the use of at least one PARP inhibitor or a pharmaceutically acceptable thereof for:
the treatment of Group 1 pulmonary arterial hypertension (PAH) in a subject in need of such treatment;
for reducing medial thickness of the pulmonary arteries of a subject suffering from Group 1 pulmonary arterial hypertension, or
for inhibiting or reducing Pulmonary Artery Smooth Muscle (PASMC) proliferation and resistance to apoptosis through a NFAT-dependent mechanism of a subject in need of such inhibiting or reducing.

One embodiment of the invention relates to the use at least one PARP inhibitor or a pharmaceutically acceptable salt thereof for the treatment of Group 1 pulmonary arterial hypertension (PAH) in a subject, including a human, in need of such treatment.

One embodiment of the invention relates to a pharmaceutical composition comprising at least one PARP inhibitor or a pharmaceutically acceptable thereof for the treatment of Group 1 pulmonary arterial hypertension.

One embodiment of the invention relates to a method of treating a subject suffering from Group 1 pulmonary arterial hypertension (PAH) which comprises administering to a said human in need of such treatment a dose effective against PAH of at least one PARP inhibitor or a pharmaceutically acceptable salt thereof.

One embodiment of the invention relates to a method of identifying a patient at risk of Group 1 PAH comprising identifying the level of PARP in a sample of a subject and making a decision regarding identifying the patient at risk of Group 1 PAH, wherein the decision is made based on the level of expression of PARP in the patient compared to a reference level.

One embodiment of the invention relates to a method of diagnosing group 1 pulmonary arterial hypertension (PAH) in a subject comprising determining the PARP level in a biological sample of the subject wherein an elevated PARP level compared to a reference sample indicates that the subject suffers from group 1 PAH.

One embodiment of the invention relates to a method of diagnosing group 1 pulmonary arterial hypertension (PAH) in a subject comprising determining the PARP regulation in a biological sample of the subject wherein an up-regulated PARP level compared to a reference sample indicates that the subject suffers from group 1 PAH.

One embodiment of the invention relates to a method for evaluating the likelihood group 1 pulmonary arterial hypertension (PAH) in a subject comprising:
comparing a PARP level in a biological sample from a subject to be tested to a reference PARP level obtained from a healthy subject; and
determining if the level of PARP in said biological sample is different from the level of the reference PARP;
wherein determination of a difference is indicative of the likelihood of group 1 PAH in said subject to be tested.

### BRIEF DESCRIPTION OF THE FIGURES

FIG. 1: (A) The level of 8-hydroxy-desoxyguanosine (80HdG) was monitored in control PASMC and PAH-PASMC from rats. To do this, we used immunofluorescence analysis using an anti-OHdG antibody. In undamaged cells, this antibody stains mitochondrial DNA damage, but not the nucleus. PAH rat cells displayed an increase in nuclear staining corresponding to increased DNA damage, which is reversed by the addition of ABT-888. (B) Quantification of unstained nuclei (negative) versus stained nuclei (stained) counted in rat cells stained for 80HdG. (C) Quantification of 80HdG positive cells in the human PAH patient versus control patient. (D) Quantification of 80HdG positive cells in the Control patient with or without PDGF treatment.

FIG. 2: (A) Quantification of g-H2AX foci formation in rat PASMC cells (untreated and treated with ABT-888). The number of nuclear foci were counted and classified as depicted (no foci, green; 0-10 foci, yellow; more than 10 foci, red). (B) Quantification of 53BP1 foci formation in rat PASMC cells (untreated and treated with ABT-888). The number of nuclear foci were counted and classified as depicted (no foci, green; 0-10 foci, yellow; more than 10 foci, red). (C) The oxidative damage was measured in PAH-PASMC compared to control cell. As predicted, PAH-PASMC have more oxidative DNA damage than the control cells. The oxidative DNA damage upregulation is associated with the increase of the PARP-1 expression.

FIG. 3: PARP-1 and pADPr expression Human PASMC were isolated from patient with or without PAH. All the cells were used after the third passage. (A) (B) Using qRT-PCR and western blot, PARP-1 expression was quantified in human PAH-PASMC and control PASMC. (C) (D) pADPr quantification in rat and human PASMC cells: pADP-ribosylation was monitored on whole cell extracts (untreated or treated with ABT-888, as indicated), using an antibody recognizing PAR.

FIGS. 4A, B and C: PARP inhibition decreases proliferation and increase apoptosis. Role of PARP in proliferation was demonstrated using PCNA staining and the measure of calcium concentration by Fluo3-AM (immunofluorescence). As shown, PAH cells are more proliferative than control cell, and PARP inhibition by ABT888 treatment decrease significantly the PCNA positive cell as well as the calcium concentration. TUNEL staining was used to measured apoptosis. The decreased in serum starvation induced apoptosis in PAH-PASMC is restored after PARP inhibition (ABT888).

FIG. 5 describes the integrative genomics approach taken to understand PARP inhibitor mode of action in PAH. (A) Transcriptomic data analysis from healthy (N=2) and PAH (N=2) patients. (B) PARP1, PARP2 and Poly ADP ribose interacting proteins (PARP interactome) generated by mass spectrometry. (C) Proteins over-expressed significantly (p<0.01, fold change>1.5) that are present in the PARP interactome. Stars (*) mark proteins known to be implicated in glycolysis. (D) Pathway enrichment analysis performed on the list of all the genes presented in Figure C.

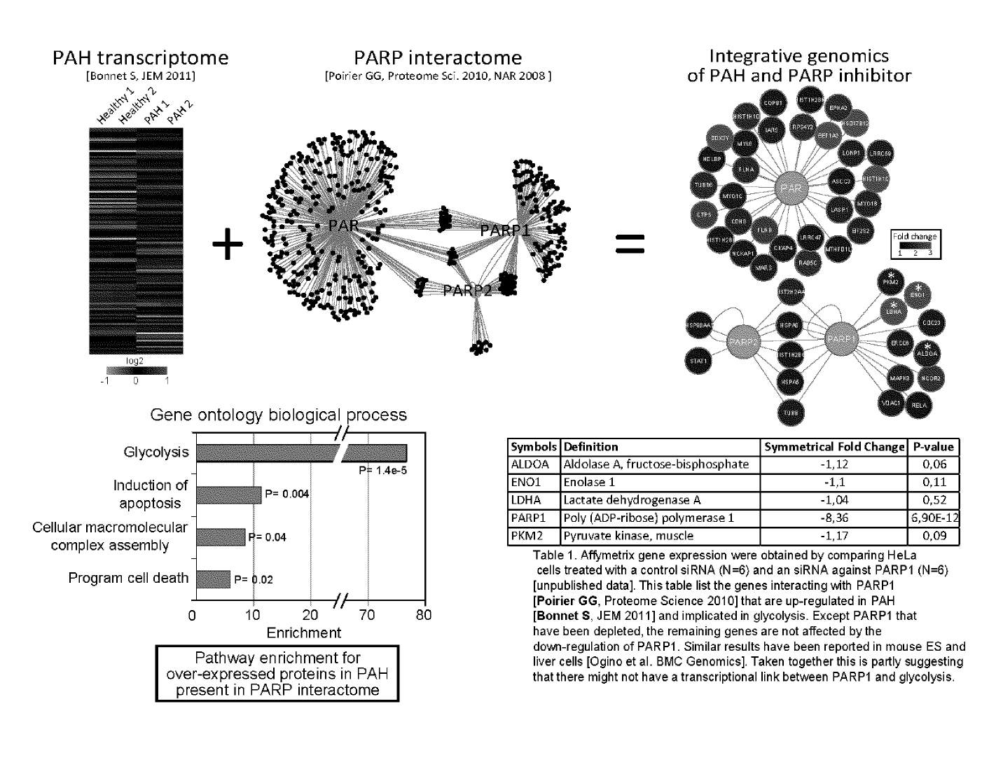

FIGS. 6A and B: PARP inhibition reverses NFAT/HIF-1 activation NFAT activation was measured by immunofluorescence, and the nuclear translocation assay. NFAT activation is significantly increased in PAH PASMC. PARP inhibition by ABT-888 significantly decrease its activation. Similarly to NFAT, HIF-1alpha activation is increased in PAH-PASMC, PARP inhibition significantly decreases HIF-1 activation in PAH-PASMC.

FIG. 7: PARP inhibition reverses PAH in MCT rats (A) (B) (C) PARP inhibition was realized in vivo using ABT888 administrated per os. As shown, after 2 weeks of treatment, mean PA pressure which is increase in PAH rats is significantly decreased in rats with treatment. The right ventricular hypertrophy (evaluated by the fulton ratio) that occur secondary to the increase in PA pressure is also decrease after ABT888 treatment. The decrease in PA pressure seen in ABT-888 treated animals is associated to a significant reduction in PA remodelling (H&E coloration). (D) (E) The decrease in PA remodelling in ABT-888 treated animals is associated to a significant decrease in PASMC proliferation (PCNA) and an increase in PASMC apoptosis.

FIG. 8: PARP-1 expression is increased in PAH human lungs PARP-1 mRNA expression (qRT-PCR) and protein expression (% of PARP-1 within the nucleus measured by PARP-1 and DAPI co-localization in immunofluorescence) and its activation levels (amount of poly-ADP-ribose polymer measured by immunofluorescence) were measured in: i) distal PA (<600 μm) in lung biopsies slides from 8 individuals with non-familial PAH compared to biopsies from 8 individuals without pulmonary hypertension (FIG. 8).

FIG. 9: PARP inhibition reverses PAH development PARP-1 inhibition decreased right ventricle wall thickness when compared to MCT-PAH rats treated with vehicle (FIGS. 9A and B). These findings were invasively confirmed by direct PA pressure measurements and measurements of the RV/LV+S weigh ratio (Fulton index) (FIGS. 9C and D). To determine whether PARP-1 inhibition can reduce pulmonary artery remodelling in MCT-PAH rats, we measured distal PA medial wall thickness. We observed that rats treated with the PARP-1 inhibitor displayed a significant reduction in medial thickness in small (≦300 μm) and medium-sized (≦600 μm) pulmonary arteries (FIG. 9E).

FIG. 10: ABT-888 treatment significantly decreases PARP-1 activity and expression in vivo, confirming the efficiency of our therapeutic strategy.

FIG. 11: PARP-1 increases PAAT in Sugen rats model.

The following abbreviations are used herein:

PAH: Pulmonary Arterial Hypertension

PA: Pulmonary Artery

PASMC: Pulmonary Artery Smooth Muscle

PAEC: Pulmonary Artery Endothelial Cells

PARP(s): Poly(ADP-ribose) polymerase(s)

PARP-1: Poly(ADP-ribose) polymerase 1

PAAT: Pulmonary Artery Acceleration Time

MCT: Monocrotaline

The present study is the first providing evidence and mechanistic approaches of PARP-1 implication in the etiology of human PAH. No studies have studied the putative implication of PARP-1 in PAH. Using both in vitro (human and rats PAH-PASMC) and in vivo (monocrotaline-induced PAH in rats) the present inventors have shown that PARP-1 is upregulated in PAH accounting for PAH-PASMC proliferation and resistance to apoptosis through a NFAT-dependent mechanism.

The present inventors have demonstrated that orally administrated PARP-1 inhibitors in rats with established PAH reverses distal PA's remodelling and decreases pulmonary arterial blood pressure in the gold standard monocrotaline induced PAH model. These effects were associated with a decrease in NFATc2 activation, PASMC proliferation and resistance to apoptosis, thus confirming the in vitro findings in human PAH-PASMC. Monocrotaline animal model is a well accepted model and is commonly used to study pulmonary hypertension. It has largely contributed to the development of new therapeutics for PAH over the last decade. A variety of therapeutic strategies has been tested in monocrotaline based models. Several of these approaches were also shown to be effective in PAH patients, and clinically proven treatments also work in this animal model.

The present inventors have found that aberrantly expressed and activated PARP-1 plays a critical role in the etiology of human PAH. The present inventors have demonstrated in vitro and in vivo that PARP-1 can be therapeutically targeted leading to a decrease of proliferation, vascular remodelling and pulmonary arterial blood pressure.

In one aspect, the present invention concerns PARP inhibitors or pharmaceutically acceptable salts thereof, for use in treating Group 1 pulmonary arterial hypertension (PAH).

In one aspect, the present invention concerns a method of treating a subject suffering from Group 1 pulmonary arterial hypertension, by administering to a said subject in need of such treatment an effective dose of at least one PARP inhibitors or pharmaceutically acceptable salts thereof.

In one aspect, the present invention concerns a method of treating warm-blooded animals including humans suffering from Group 1 pulmonary arterial hypertension, by administering to a said animal in need of such treatment an effective dose of at least one PARP inhibitors or pharmaceutically acceptable salts thereof.

The term “Group 1 Pulmonary Arterial Hypertension” as used herein refers the Venice Clinical Classification of Pulmonary Hypertension (2003). Group 1 PAH is a disease of the pulmonary vasculature, defined by an elevated pulmonary vascular resistance, leading to right heart failure and premature death. PAH is characterized by enhanced pulmonary artery smooth muscle and endothelial cells proliferation and suppressed apoptosis within pulmonary artery wall.

More specifically, pulmonary arterial hypertension of Group 1 includes:
1.1. Idiopathic (IPAH)
1.2. Familial (FPAH)
1.3. Associated with (APAH)
   1.3.1. Collagen vascular disease
   1.3.2. Congenital systemic-to-pulmonary shunts
   1.3.3. Portal hypertension
   1.3.4. HIV infection
   1.3.5. Drugs and toxins
   1.3.6. Other (thyroid disorders, glycogen storage disease, Gaucher disease, hereditary hemorrhagic telangiectasia, hemoglobinopathies, myeloproliferative disorders, splenectomy)
1.4. Associated with significant venous or capillary involvement
   1.4.1. Pulmonary veno-occlusive disease (PVOD)
   1.4.2. Pulmonary capillary hemangiomatosis (PCH)
1.5. Persistent pulmonary hypertension of the newborn.

In one aspect the present invention concerns a method of treating a subject, including a human, suffering from:

(a) idiopathic or primary pulmonary hypertension,

(b) familial hypertension,

(c) pulmonary hypertension secondary to, but not limited to, connective tissue disease, congenital heart defects (shunts), pulmonary fibrosis, portal hypertension, HIV infection, sickle cell disease, drugs and toxins (e.g., anorexigens, cocaine), chronic hypoxia, chronic pulmonary obstructive disease, sleep apnea, and schistosomiasis,

(d) pulmonary hypertension associated with significant venous or capillary involvement (pulmonary veno-occlusive disease, pulmonary capillary hemangiomatosis),

(e) secondary pulmonary hypertension that is out of proportion to the degree of left ventricular dysfunction, or

(f) persistent pulmonary hypertension in newborn babies, which comprises administering to said human in need of such treatment a dose effective against the respective disorder of at least one PARP inhibitors or pharmaceutically acceptable salts thereof. A method of treating a human suffering from pulmonary arterial hypertension (PAH) which comprises administering to said subject in need of such treatment a dose effective against PAH of at least one PARP inhibitor or a pharmaceutically acceptable salt thereof.

One embodiment of the present invention relates to the use of at least one PARP inhibitors or pharmaceutically acceptable salts thereof for reducing medial thickness of the pulmonary arteries of a subject (e.g. warm-blooded animals including humans) suffering from Group 1 pulmonary arterial hypertension, by administering to a said animal in need of such treatment an effective dose.

One embodiment of the present invention relates to the use of at least one PARP inhibitors or pharmaceutically acceptable salts thereof for inhibiting or reducing PAH-PASMC proliferation and resistance to apoptosis through a NFAT-dependent mechanism a subject (e.g. warm-blooded animals including humans) by administering to a said subject in need of such treatment an effective dose.

One embodiment of the present invention relates to the use of at least one PARP inhibitors or pharmaceutically acceptable salts thereof for reducing medial thickness of the pulmonary arteries of warm-blooded animals including humans suffering from Group 1 pulmonary arterial hypertension, by administering to a said animal in need of such treatment an effective dose.

One embodiment of the present invention relates to the use of an effective dose of at least one PARP inhibitors or pharmaceutically acceptable salts thereof for reducing medial thickness of the pulmonary arteries of a subject (e.g. warm-blooded animals including humans) suffering from Group 1 pulmonary arterial hypertension.

One embodiment of the present invention relates to the use of an effective dose of at least one PARP inhibitors or pharmaceutically acceptable salts thereof for inhibiting or reducing PAH-PASMC proliferation and resistance to apoptosis through a NFAT-dependent mechanism a subject (e.g. warm-blooded animals including humans).

One embodiment of the present invention relates to the use of an effective dose of at least one PARP inhibitors or pharmaceutically acceptable salts thereof for reducing medial thickness of the pulmonary arteries of warm-blooded animals including humans suffering from Group 1 pulmonary arterial hypertension.

As used herein, the term “subject or patient” refers to any subject susceptible of suffering or suffering from Group 1 PAH. Specifically, such a subject may be, but not limited to, human, an animal (e.g. cat, dog, cow, horse, etc.). More specifically, the subject consists of a human.

The term “treating or treatment” as used herein refers to curative and prophylactic treatment of Group 1 PAH.

The term “curative” as used herein means efficacy in treating on going episodes of group 1 PAH.

The term “prophylactic” as used herein means the prevention of the onset or recurrence of group 1 PAH.

The status of patient suffering from PAH can be assessed according to World Health Organization (WHO) classification (modified after the New York Association Functional Classification) as detailed below:

Class I—Patients with pulmonary hypertension but without resulting limitation of physical activity. Ordinary physical activity does not cause undue dyspnea or fatigue, chest pain or near syncope.

Class II—Patients with pulmonary hypertension resulting in slight limitation of physical activity. They are comfortable at rest. Ordinary physical activity causes undue dispend or fatigue, chest pain or near syncope.

Class III—Patients with pulmonary hypertension resulting in marked limitation of physical activity. They are comfortable at rest. Less than ordinary activity causes undue dyspnea or fatigue, chest pain or near syncope.

Class IV—Patients with pulmonary hypertension with inability to carry out any physical activity without symptoms. These patients manifest signs of right heart failure. Dyspnea and/or fatigue may even be present at rest. Discomfort is increased by any physical activity.

The term “PARP inhibitor” as used herein refers to an inhibitor or antagonist of Poly(ADP-ribose) polymerases (PARP 1 and/or PARP2) activity. A PARP inhibitor or antagonist is a compound that selectively inhibits the activity of PARP and refers to a compound that when administered to a subject the PARP activity within the subject is altered, preferably reduced. A drug also able to decrease PARPs expression is also considered as PARP inhibitor. PARP is activated when Poly ADP ribose polymer is increased. In one embodiment, a prodrug of a PARP inhibitor is administered to a subject that is converted to the compound in vivo where it inhibits PARP.

The PARP inhibitor may be any type of compound. For example, the compound may be a small organic molecule or a biological compound such as an antibody or an enzyme. Example of PARP inhibitors are described in Penning, Current Opinion In Drug Discovery & Development 2010 13 (5): 577-586. A person skilled in the art can easily determine whether a compound is capable of inhibiting PARP activity. Assays for evaluating PARP activity are for example, described in Poly(ADP-ribose) (PAR) polymer is a death signal (Andrabi S A et al., 2006). PARP inhibition may be determined using conventional methods, including for example dot blots (Affar E B et al., Anal Biochem. 1998; 259(2):280-3), and BER assays that measure the direct activity of PARP to form poly ADP-ribose chains for example by using radioactive assays with tritiated substrate NAD or specific antibodies to the polymer chains formed by PARP activity (K. J. Dillon et al, Journal of Biomolecular Screening, 8(3): 347-352 (2003).

Examples of compounds which are known PARP inhibitors and which may be used in accordance with the invention include compounds and derivatives thereof from the class of Nicotinamides, Benzamides, Isoquinolinones, Dihydroisoquinolinones, Benzimidazoles, indoles, Phthalazin-1 (2H)-ones, quinazolinones, Isoindolinones, Phenanthridines, phenanthhdinones, Benzopyrones, Unsaturated hydroximic acid derivatives and Pyridazines.

Examples of compounds which are known PARP inhibitors and which may be used in accordance with the invention include:

1 . Nicotinamides, such as 5-methyl nicotinamide and O-(2-hydroxy-3-piperidino-propyl)-3-carboxylic acid amidoxime, and analogues and derivatives thereof.

2. Benzamides, including 3-substituted benzamides such as 3-aminobenzamide, 3-hydroxybenzamide, 3-nitrosobenzamide, 3-methoxybenzamide and 3-chloroprocainamide, and 4-aminobenzamide, 1,5-di[(3-carbamoylphenyl)aminocarbonyloxy]pentane, and analogues and derivatives thereof.

3. Isoquinolinones and Dihydroisoquinolinones, including 2H-isoquinolin-1-ones, 3H-quinazolin-4-ones, 5-substituted dihydroisoquinolinones such as 5-hydroxy dihydroisoquinolinone, 5-methyl dihydroisoquinolinone, and 5-hydroxy isoquinolinone, 5-amino isoquinolin-1-one, 5-dihydroxyisoquinolinone, 3,4 dihydroisoquinolin-1 (2H)-ones such as 3,4dihydro-5-methoxy-isoquinolin-1 (2H)-one and 3,4 dihydro-5-methyl-1 (2H)isoquinolinone, isoquinolin-1 (2H)-ones, 4,5-dihydro-imidazo[4,5,1-ij]quinolin-6-ones, 1,6,-naphthyridine-5(6H)-ones, 1,8-naphthalimides such as 4-amino-1,8-naphthalimide, isoquinolinone, 3,4-dihydro-5-[4-1 (1-piperidinyl) butoxy]-1(2H)-isoquinolinone, 2,3-dihydrobenzo[de]isoquinolin-1-one, 1-1 1b-dihydro-[2H]benzopyrano[4,3,2-de]isoquinolin-3-one, and tetracyclic lactams, including benzpyranoisoquinolinones such as benzopyrano[4,3,2-de]isoquinolinone, and analogues and derivatives thereof.

4. Benzimidazoles and indoles, including benzoxazole-4-carboxamides, benzimidazole-4-carboxamides, such as 2-substituted benzoxazole 4-carboxamides and 2-substituted benzimidazole 4-carboxamides such as 2-aryl benzimidazole 4-carboxamides and 2-cycloalkylbenzimidazole-4-carboxamides including 2-(4-hydroxphenyl)benzimidazole 4-carboxamide, quinoxalinecarboxamides, imidazopyridinecarboxamides, 2-phenylindoles, 2-substituted benzoxazoles, such as 2-phenyl benzoxazole and 2-(3-methoxyphenyl) benzoxazole, 2-substituted benzimidazoles, such as 2-phenyl benzimidazole and 2-(3-methoxyphenyl) benzimidazole, 1,3,4,5 tetrahydro-azepino[5,4,3-cd]indol-6-one, azepinoindoles and azepinoindolones such as 1,5 dihydro-azepino[4,5,6-cd]indolin-6-one and dihydrodiazapinoindolinone, 3-substituted dihydrodiazapinoindolinones,such as 3-(4-thfluoromethyl phenyl)-dihydrodiazapinoindolinone, tetrahydrodiazapinoindolinone and 5,6,-dihydroimidazo[4,5,1-j,k][1,4]benzodiazopin-7(4H)-one, 2-phenyl-5,6-dihydro-imidazo[4,5,1-jk][1,4]benzodiazepin-7(4H)-one and 2,3, dihydro-isoindol-1-one, and analogues and derivatives thereof.

5. Phthalazin-1 (2H)-ones and quinazolinones, such as 4-hydroxyquinazoline, phthalazinone, 5-methoxy-4-methyl-1 (2) phthalazinones, 4-substituted phthalazinones, 4-(1-piperazinyl)-1 (2H)-phthalazinone, tetracyclic benzopyrano[4,3,2-de]phthalazinones and tetracyclic indeno [1,2,3-de]phthalazinones and 2-substituted quinazolines, such as 8-hydroxy-2-methylquinazolin-4-(3H) one, tricyclic phthalazinones and 2-aminophthalhydrazide, and analogues and derivatives thereof.

6. Isoindolinones and analogues and derivatives thereof

7. Phenanthridines and phenanthridinones, such as 5[H]phenanthridin-6-one, substituted 5[H]phenanthridin-6-ones, especially 2-, 3- substituted 5[H]phenantridin-6-ones and sulfonamide/carbannide derivatives of 6(5H)phenanthridinones, thieno[2,3-c]isoquinolones such as 9-annino thieno[2,3-c]isoquinolone and 9-hydroxythieno[2,3-c]isoquinolone, 9-methoxythieno[2, 3-c]isoquinolone, and N-(6-oxo-5,6-dihydrophenanthridin-2-y]-2-(N,N-dimethylannino}acetannide, substituted 4,9-dihydrocyclopenta[lmn]phenanthridine-5-ones, and analogues and derivatives thereof.

8. Benzopyrones such as 1,2-benzopyrone, 6-nitrosobenzopyrone, 6-nitroso 1,2-benzopyrone, and 5-iodo-6-aminobenzopyrone, and analogues and derivatives thereof.

9. Unsaturated hydroximic acid derivatives such as O-(3-piperidino-2-hydroxy-1-propyl)nicotinic amidoxime, and analogues and derivatives thereof.

10. Pyridazines, including fused pyridazines and analogues and derivatives thereof.

11 . Other compounds such as caffeine, theophylline, and thymidine, and analogues and derivatives thereof.

Additional PARP inhibitors are described for example in WO2009093032, WO2009004356, WO2006078503 WO2006078711, WO200642638, WO2006024545, WO2006003150, WO2006003148, WO2006003147, WO2006003146, WO2004043959, WO2005123687, WO2005097750, WO2005058843, WO2005054210, WO2005054209, WO2005054201, US2005054631, WO2005012305, WO2004108723, WO2004105700, US2004229895, WO2004096793, WO2004096779, WO2004087713, WO2004048339, WO2004024694, WO2004014873, U.S. Pat. No. 6,635,642, U.S. Pat. No. 5,587,384, WO2003080581, WO2003070707, WO2003055865, WO2003057145, WO2003051879, U.S. Pat. No. 6,514,983, WO2003007959, U.S. Pat. No. 6,426,415, WO2003007959, WO 2002036599, WO2002094790, WO2002068407, U.S. Pat. No. 6,476,048, WO2001090077, WO2001085687, WO2001085686, WO2001079184, WO2001057038, WO2001023390, WO2001021615, WO2001016136, WO2001012199, WO9524379, Banasik et al. J. Biol. Chem., 267:3, 1569-75 (1992), Banasik et al. Molec. Cell. Biochem. 138:185-97 (1994)), Cosi (2002) Expert Opin. Ther. Patents 12 (7), and Southan & Szabo (2003) Curr Med Chem 10:321 -340, and references therein.

Other examples of compounds which are known PARP inhibitors include the hydrochloride salt of /V-(-oxo-5,6-dihydro-phenanthridin-2-yl)-/V,/V-dimethylacetamide and other analogues or similar compounds, such as INO-1001 that show PARP inhibition.

In one embodiment, PARP inhibitors include NU1025, ABT-888 (Veliparib), Olaparib (was AZD-2281), CEP 9722, MK4827, AG014699, Iniparib (previously BSI 201), LT-673, 3-aminobenzamide and E7016.

In one embodiment, the PARP inhibitor is ABT-888 represented by the formula:

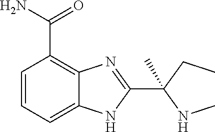 [SMILES: CC[C@@](C)(NC)c1nc2c(C(N)=O)cccc2[nH]1]

or a pharmaceutically acceptable salt thereof.

It is noted in that the present invention is intended to encompass all pharmaceutically acceptable ionized forms (e.g., salts) and solvates (e.g., hydrates) of the PARP inhibitors, regardless of whether such ionized forms and solvates are specified since it is well known in the art to administer pharmaceutical agents in an ionized or solvated form. It is also noted that unless a particular stereochemistry is specified, recitation of a compound is intended to encompass all possible stereoisomers (e.g., enantiomers or diastereomers depending on the number of chiral centers), independent of whether the compound is present as an individual isomer or a mixture of isomers.

There is also provided pharmaceutically acceptable salts of the PARP inhibitors. By the term pharmaceutically acceptable salts are meant those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Salts derived from amino acids are also included (e.g. L-arginine, L-Lysine). Salts derived from appropriate bases include alkali metals (e.g. sodium, lithium, potassium) and alkaline earth metals (e.g. calcium, magnesium).

With regards to pharmaceutically acceptable salts, see also the list of FDA approved commercially marketed salts listed in Table I of Berge et al., Pharmaceutical Salts, J. of Phar. Sci., vol. 66, no. 1, January 1977, pp. 1-19.

It will be appreciated by those skilled in the art that the PARP inhibitor can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

It will further be appreciated by those skilled in the art that the PARP inhibitor can exist in different solvate forms, for example hydrates. Solvates of the PARP inhibitor may also form when solvent molecules are incorporated into the crystalline lattice structure of the compound molecule during the crystallization process.

It will be appreciated that the amount of a PARP inhibitor required for use in treatment will vary not only with the particular compound selected but also with the route of administration, the nature of the condition for which treatment is required and the age and condition of the patient and will be ultimately at the discretion of the attendant physician. In general however a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

The PARP inhibitor is conveniently administered in unit dosage form; for example containing 5 to 2000 mg, 10 to 1500 mg, conveniently 20 to 1000 mg, most conveniently 50 to 700 mg of active ingredient per unit dosage form.

When PARP inhibitor or pharmaceutically acceptable salts thereof are used in combination with a second therapeutic agent active against Group 1 PAH the dose of each compound may be either the same as or differ from that when the compound is used alone. Appropriate doses will be readily appreciated by those skilled in the art.

While it is possible that, for use in therapy, the PARP inhibitor may be administered as the raw chemical it is preferable to present the active ingredient as a pharmaceutical composition. The invention thus further provides a pharmaceutical composition comprising the PARP inhibitor or a pharmaceutically acceptable salt thereof together with one or more pharmaceutically acceptable carriers therefore and, optionally, other therapeutic and/or prophylactic ingredients. The carrier(s) must be “acceptable” in the sense of being compatible with the other ingredients of the formulation and not deleterious to the recipient thereof.

Pharmaceutical compositions include those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The compositions may, where appropriate, be conveniently presented in discrete dosage units and may be prepared by any of the methods well known in the art of pharmacy. All methods include the step of bringing into association the active with liquid carriers or finely divided solid carriers or both and then, if necessary, shaping the product into the desired composition.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The PARP inhibitor may also be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the PARP inhibitor may be formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Compositions suitable for topical administration in the mouth include lozenges comprising active ingredient in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical compositions suitable for rectal administration wherein the carrier is a solid are for example presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

Compositions suitable for vaginal administration may be presented as pessaries, tampons, creams, gels, pastes, foams or sprays containing in addition to the active ingredient such carriers as are known in the art to be appropriate.

For intra-nasal administration the compounds or combinations may be used as a liquid spray or dispersible powder or in the form of drops. Drops may be formulated with an aqueous or non-aqueous base also comprising one more dispersing agents, solubilizing agents or suspending agents. Liquid sprays are conveniently delivered from pressurized packs.

For administration by inhalation the compounds or combinations are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the compounds or combinations may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

As used herein, the expression “an acceptable carrier” means a vehicle for containing the compounds obtained by the method of the invention that can be administered to a subject without adverse effects. Suitable carriers known in the art include, but are not limited to, gold particles, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

In a further embodiment, the invention relates to a method of treating a warm-blooded animal, especially a human, suffering from pulmonary hypertension, especially pulmonary arterial hypertension, comprising administering to the animal a combination which comprises (a) at least one PARP inhibitor or a pharmaceutically acceptable salt thereof and (b) at least one compound selected from compounds indicated for the treatment of pulmonary arterial hypertension, such as calcium channel antagonists, e.g. nifedipine, e.g. 120 to 240 mg/d, or diltiazem, e.g. 540 to 900 mg/d, prostacyclin, the prostacyclin analogues iloprost, flolan and treprostinil, adenosine, inhaled nitric oxide, anticoagulants, e.g. warfarin, digoxin, endothelin receptor blockers, e.g. bosentan, phosphodiesterease inhibitors, e.g. sildenafil, norepinephrine, angiotensin-converting enzyme inhibitors e.g. enalapril or diuretics; a combination comprising (a) and (b) as defined above and optionally at least one pharmaceutically acceptable carrier for simultaneous, separate or sequential use, in particular for the treatment of pulmonary arterial hypertension; a pharmaceutical composition comprising such a combination; the use of such a combination for the preparation of a medicament for the delay of progression or treatment of pulmonary arterial hypertension; and to a commercial package or product comprising such a combination.

When the combination partners employed in the combinations as disclosed herein are applied in the form as marketed as single drugs, their dosage and mode of administration can take place in accordance with the information provided on the package insert of the respective marketed drug in order to result in the beneficial effect described herein, if not mentioned herein otherwise.

As used herein, the term “sample” refers to a variety of sample types obtained from a subject and can be used in a diagnostic assay. The definition encompasses blood and other liquid samples of biological origin, solid tissue samples such as a biopsy specimen or tissue culture or cells derived therefrom. In some embodiments, the sample is selected from the group consisting of human normal sample, tumor sample, hair, blood, cell, tissue, organ, brain tissue, blood, serum, sputum, saliva, plasma, nipple aspirant, synovial fluid, cerebrospinal fluid, sweat, urine, fecal matter, pancreatic fluid, trabecular fluid, cerebrospinal fluid, tears, bronchial lavage, swabbing, bronchial aspirant, semen, prostatic fluid, precervicular fluid, vaginal fluids, and pre-ejaculate. In a further aspect, the sample is lung or blood.

As used herein, the expression “reference marker” or “reference level” refers to a marker or marker level present in a healthy subject i.e. not suffering group 1 PAH.

The expression “increased risk” when used in conjunction with “Group 1 Pulmonary Arterial Hypertension” means to denote the probability that Group 1 Pulmonary Arterial Hypertension will develop in the subject.

As used herein, the expression “PARP marker” refers to a PARP polypeptide or protein or to a nucleotide sequence encoding a PARP in the form of DNA or RNA.

As used herein, the expression “PARP-specific antibody” refers to antibodies that bind to one or more epitopes of PARP protein, but which do not substantially recognize and bind other molecules in a sample containing a mixed population of antigenic molecules.

One aspect of the invention relates to a method of identifying a patient at risk of group 1 PAH comprising identifying a level of PARP in a sample of a subject, making a decision regarding identifying the group 1 PAH wherein the decision is made based on the level of expression of PARP.

In some embodiments, the identification of the level of PARP comprises assay technique. In some preferred embodiments, the assay technique measures expression of PARP gene or protein. In some preferred embodiments, the level of PARP is up-regulated.

The PARP polypeptide and polynucleotide encoding same contemplated by the present invention may also be used in different ways in the diagnosis of group 1 PAH.

In this connection and in a further embodiment, the present invention provides a method for evaluating the likelihood of group 1 PAH in a subject. The method comprises the following steps:
a. comparing a PARP level in a biological sample from a subject to be tested to a reference PARP level obtained from a healthy subject; and
b. determining if the level of PARP in said biological sample is different from the level of the reference PARP;

wherein determination of a difference is indicative of the likelihood of group 1 PAH in said subject to be tested.

It will be understood by one skilled in the art that the expressions “difference in levels” or “different from the level” mean that the level of PARP measured in a biological sample is higher than the level of PARP measured in the control or reference sample.

The larger is the difference between the levels of PARP, higher may be the risk of suffering or having group 1 PAH.

As one skilled in the art will appreciate, the comparison between PARP levels is indicative of the subject's risk of suffering or having Group 1 PAH. When the levels of PARP are substantially identical, the subject's risk of suffering or having group 1 PAH may be low. However, larger the difference in the levels of the PARP is, higher may be the risk of suffering or having group 1 PAH.

As one skilled in the art may appreciate, the measurement of PARP level may be performed by detecting and quantifying the PARP protein/polypeptide itself and/or the polynucleotide encoding the same within a biological sample. In the case where the PARP to be measured is a protein or a polypeptide, the detection of PARP may involves a detecting agent, which may be, for instance, a specific antibody such as a purified monoclonal or polyclonal antibody raised against PARP protein or a polypeptide thereof. In such a case, the determination of PARP marker level is achieved by contacting a PARP specific antibody with the biological sample under suitable conditions to obtain a PARP-antibody complex.

Once detected, PARP may be quantified in accordance with biochemical assays known by the skilled person in the art of biochemistry and/or analytical chemistry. Particularly, PARP level may be quantified by, but not limited to, immunoassays such as enzyme-linked immunosorbent assay (ELISA), radioimmunoassay (RIA), magnetic immunoassay (MIA) or immunoblot (Western blot).

Where the detection of a PARP nucleotide sequence is advantageously sought, such may be achieved, for instance, by a genetic detection means, such as a nucleic acid hybridization process (e.g. Southern blots and Northern blots) or a nucleic acid amplifying process (e.g. polymerase chain reaction (PCR)) so as to detect and quantify specific regions of a RNA or DNA strand of the PARP nucleotide sequence.

The present invention further provides kits for use within any of the above diagnostic methods. Such kits typically comprise two or more components necessary for performing a diagnostic assay. Components may be compounds, reagents, containers and/or equipment. For example, one container within a kit may contain an antibody or fragment thereof that specifically binds to a PARP polypeptide contemplated by the present invention. One or more additional containers may enclose elements, such as reagents or buffers, to be used in the assay.

Alternatively, a kit may be designed to detect the level of mRNA or cDNA encoding PARP protein in a biological sample. Such kits generally comprise at least one oligonucleotide probe or primer, as described above, that hybridizes to a polynucleotide encoding PARP protein. Such an oligonucleotide may be used, for example, within a PCR or hybridization assay. Additional components that may be present within such kits include a second oligonucleotide and/or a reagent or container to facilitate the detection or quantification of a polynucleotide encoding PARP protein.

The present invention will be more readily understood by referring to the following example. The examples are illustrative of the wide range of applicability of the present invention and are not intended to limit its scope. Modifications and variations can be made therein without departing from the spirit and scope of the invention. Although any methods and materials similar or equivalent to those described herein can be used in the practice for testing of the present invention, the following methods and materials are described. The issued patents, published patent applications, and references that are cited herein are hereby incorporated by reference to the same extent as if each was specifically and individually indicated to be incorporated by reference. In the case of inconsistencies, the present disclosure will prevail.

### EXAMPLES AND RESULTS

### Example 1

PARP-1 is Upregulated in Human and Rodent PAH-PASMC.

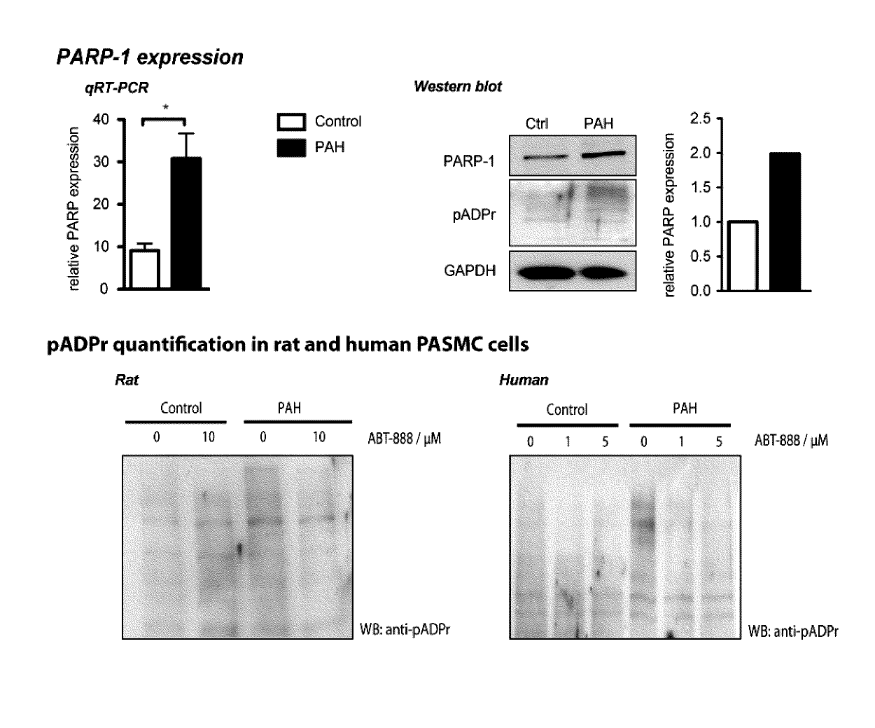

The involvement of PARP in PAH was confirmed by monitoring whether PARP-1 is aberrantly expressed in human and rodent PAH. PASMC were isolated from distal pulmonary arteries of two non-familial PAH patients and 2 control patients; 5 fawn-Hooded rats (FHR) with established PAH and 5 FHR-BN1 known to be resistant to PAH (Bonnet et al., 2006). All these cells were cultured as previously described (Bonnet et al., 2006; McMurtry et al., 2005) (passage 6 and less). The expression and activity of PARP-1 was measured. PARP-1 upregulation in the PAH group versus the control group was confirmed by qRT-PCR in both human and rodent PASMC. Activity was measured by immunofluorescence detection of poly (ADP-ribose) polymer formation. As shown in FIG. 3, PARP-1 activity is significantly increased in both human and rodent PAH-PASMC.

### Example 2

PARP-1 Activation is Associated with PASMC Oxidative Stress, Disrupted Mitochondria and DNA Injury.

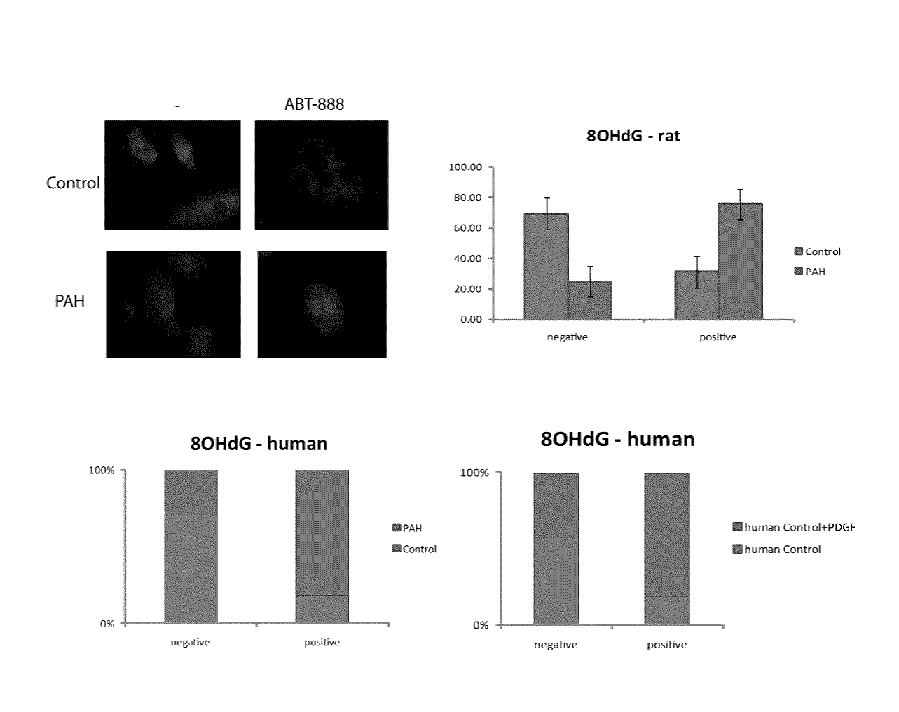

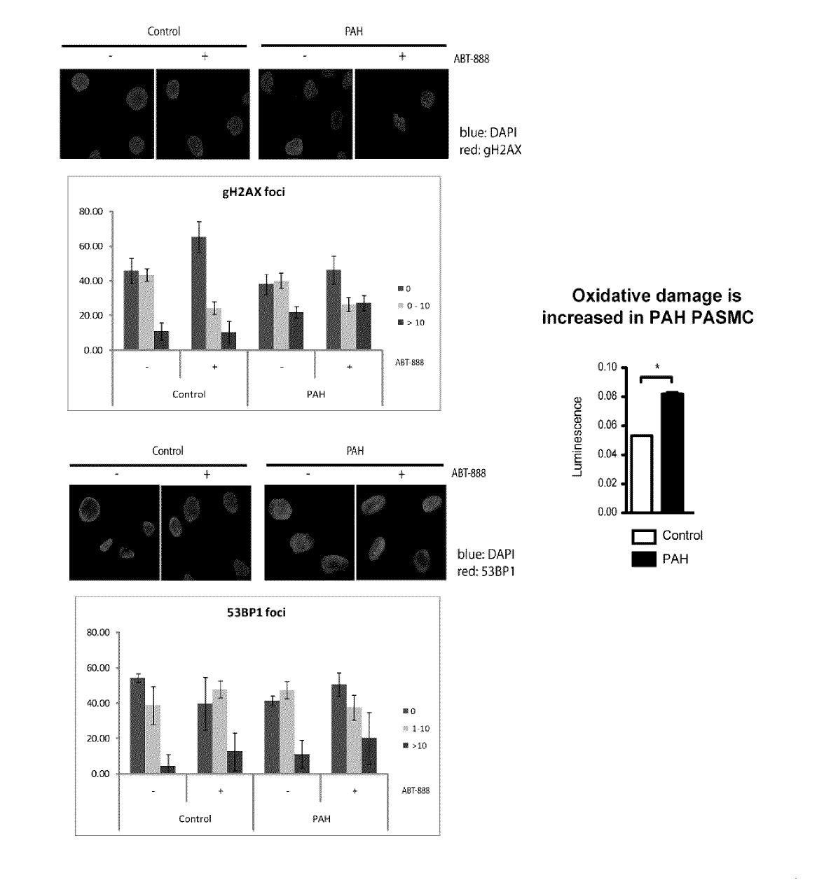

As it is well known that PARP activity is induced by DNA damage, we monitored the level of 8-hydroxy-desoxyguanosine (8OHdG) in healthy rat PASMC cells and rat PAH-PASMC. To do this, we used immunofluorescence analysis using anti-OHdG antibody. In undamaged cells, this antibody stains mitochondrial DNA damage, but not the nucleus. PAH-PASMC cells displayed an increase in nuclear staining corresponding to increased DNA damage (FIG. 1). Similarly, the anti-8OHdG nuclear staining of human PAH cells was significantly increased compared to control PASMC. PAH can be recapitulated by treatment of control cells with PDGF. Again, human control cells treated with PDGF accumulated 8OHdG compared to the untreated cells. To confirm that DNA damage is increased in PAH, 53BP1 staining was used as a surrogate marker for the accumulation of DNA double strand breaks (DSBs). PAH-PASMC showed a 2-fold increase in 53BP1 foci compared to control PASMC (FIGS. 2A&B). Furthermore, an increase in oxidative stress was observed (FIG. 2C). Finally, PARP-1 mRNA and protein levels as well as PAR polymer levels are increased in PAH-PASMC compare to control PASMC (FIG. 3) showing that the levels of PARP-1 and DNA damage are important factors contributing to PAH.

### Example 3

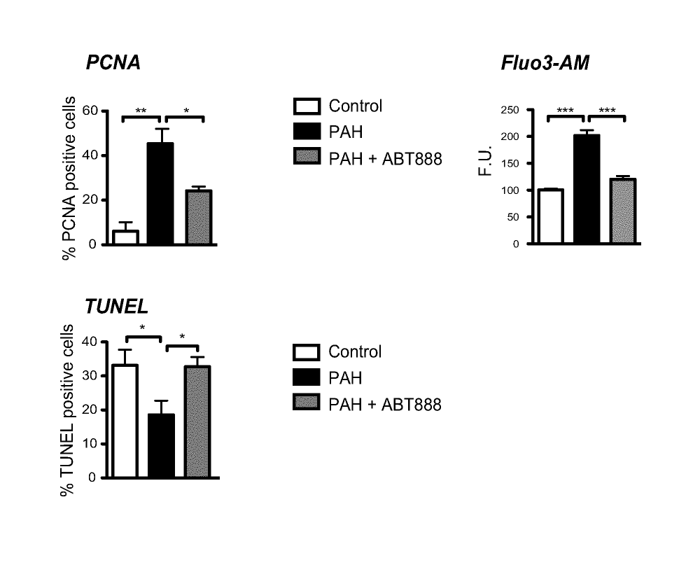

PARP-1 Promotes PASMC Proliferation and Resistance to Apoptosis (FIG. 4).

To study the effect of PARP-1 on PASMC proliferation and apoptosis in vitro, cultured human PAH-PASMCs were either exposed to 10% FBS to promote proliferation or 0.1% FBS to induce apoptosis (Bonnet et al., 2007b). When compared to control PASMCs containing a low level of PARP-1, PAH-PASMCs displayed higher cell proliferation rate and resistance to induced apoptosis. The implication of PARP-1 in regulating PASMC proliferation and apoptosis was confirmed in PAH-PASMCs, in which PARP-1 inhibition decreased proliferation and resistance to apoptosis to levels similar to those seen in control-PASMCs.

### Example 4

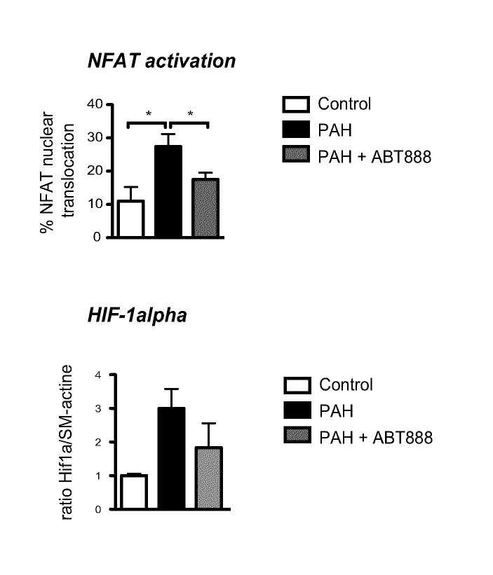

PARP-1 Upregulation Promotes the Activation of the Pro-Proliferative and Anti-Apoptotic NFAT Pathway in PAH-PASMCs. PARP Promotes NFAT and HIF (FIG. 6)

The increase in PASMC proliferation and resistance to apoptosis observed in PAH has been linked to the activation of the NFAT pathways (Bonnet et al., 2007b). The putative implication of PARP-1 in these pathways was thus investigated. As expected, we observed an increase in the activity of NFAT (increased NFATc2 nuclear translocation) in PAH-PASMCs. This finding demonstrates that an upregulation of PARP-1 leads to the activation of NFAT pathway in PAH-PASMCs. This finding is in agreement with previously published results in T-cells (Olabisi et al., 2008; Valdor et al., 2008).

### Example 5

Decreasing PARP-1 Level in PAH-PASMCs Reverses the Pro-Proliferation and Anti-Apoptotic Phenotype of PAH-PASMCs (FIG. 4).

In PAH-PASMCs, NFAT-mediated proliferation (Bonnet et al., 2007b; Wong et al., 2005) has been linked to the downregulation of K⁺channels (Bonnet and Archer, 2007; Platoshyn et al., 2000) resulting in membrane depolarization (Platoshyn et al., 2000; Yuan, 1995), opening the voltage-dependent calcium channels, thereby increasing intracellular calcium concentration ([Ca²⁺]ᵢ) (Bonnet et al., 2007a; Wong et al., 2005; Yuan, 1995). Using Fluo-3AM and PCNA we measured the effect of PARP-1 inhibition on [Ca²⁺]ᵢ and PASMCs proliferation. The PARP-1 inhibition in PAH-PASMCs decreases [Ca²⁺]ᵢ and PASMC proliferation to the level seen in control-PASMCs. To further confirm that these effects were mediated via NFAT pathway, we treated cells with the NFAT inhibitor VIVIT (Bonnet et al., 2007b). VIVIT treatment does not further decrease [Ca²⁺]ᵢ in PAH-PASMCs where the PARP-1 activity has been inhibited.

Resistance to apoptosis observed in PAH-PASMCs has been linked to mitochondrial membrane potential (ΔΨₘ) hyperpolarization, which would block the release of pro-apoptotic mediators like cytochrome c (Bonnet et al., 2007a; Bonnet et al., 2009). Using tetramethylrhodamine methyl ester (TMRM), we measured whether PARP-1 inhibition can affect mitochondrial hyperpolarization. PARP-1 inhibition in PAH-PASMCs depolarizes ΔΨₘ to a level similar to that observed in control-PASMCs. Moreover it was demonstrated that PARP-1 inhibition depolarizes PAH-PASMCs to a level similar to the one seen in VIVIT-treated PAH-PASMC. Finally, both PARP-1 and VIVIT did not have additive effects on ΔΨₘ suggesting that PARP-1 effects on ΔΨₘ is mainly mediated by NFAT.

### Example 6

PARP-1 Inhibition Reverses Monocrotaline (MCT)-Induced PAH.

In order to test if PARP-1 inhibition can reverse symptoms of PAH in the rat model, PARP-1 inhibitor was orally given to rats with established MCT-induced PAH rats (10-15 days after MCT injection). PARP-1 expression and activity were measured in the lungs of treated animals and compared to the untreated animals. The results revealed that orally available PARP-1 inhibitor significantly decreases PARP-1 activity and expression in vivo.

Longitudinal study to assess the efficacy of PARP inhibitor treatment was performed for two weeks using non-invasive measurements (Doppler echocardiography). PARP-1 inhibition in MCT-PAH rats reduced pulmonary arterial pressure as assessed by the pulmonary artery acceleration time (PAAT), a Doppler parameter linked to PA pressure (PAAT being inversely correlated to PA pressure). In addition, PARP-1 inhibition decreased right ventricle wall thickness when compared to MCT-PAH rats treated with vehicle. These findings were invasively confirmed by direct PA pressure measurements by right catheterization and measurements of the RV/LV+S weigh ratio. To determine whether PARP-1 inhibition can reduce pulmonary artery remodelling in MCT-PAH animals, we measured medial wall thickness.

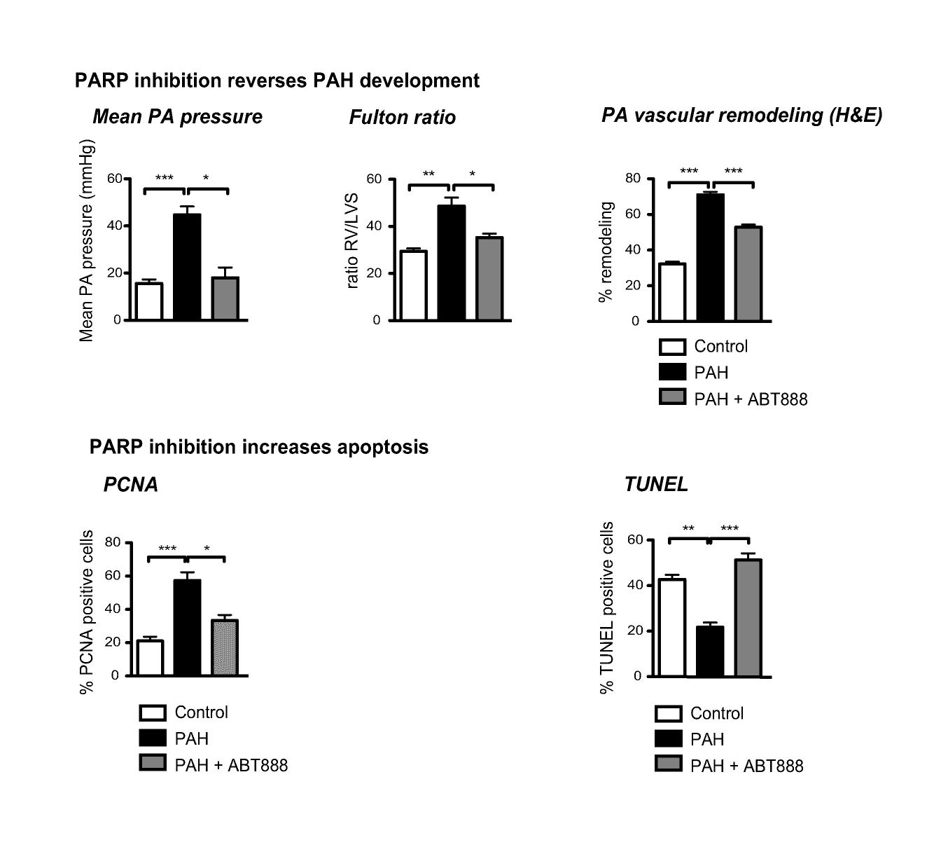

PARP-1 inhibition in MCT-PAH rats reduces pulmonary arterial pressure and decreases right ventricle wall thickness when compared to MCT-PAH rats treated with vehicle. Animals treated with PARP-1 inhibitor displayed a significant reduction in medial thickness of small (≦300 μm) and medium-sized (≦600 μm) pulmonary arteries (FIG. 7A). A significant decrease in PASMC proliferation (as assessed by PCNA distribution) and resistance to apoptosis (TUNEL) was also observed in rats treated with PARP-1 inhibitor (FIG. 7B).

### Example 7

PARP-1 Expression is Increased in PAH Human Lungs.

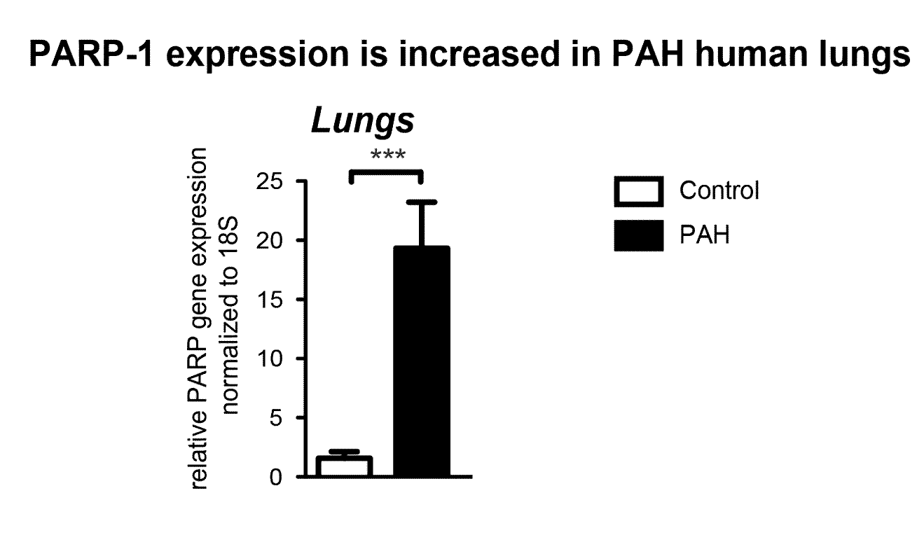

PARP upregulation in human lung from 8 PAH patients compared to 8 healthy patients was quantified by qRT-PCR. Total RNA was extracted from paraffin lung with a specific RecoverAll Total Nucleic Acid Isolation Kit (Applied Biosystems; # AM1975). PARP expression was measured with specific taqman assay (Applied Biosystems). As shown, mRNA PARP expression is significantly increased in human PAH patients compare to healthy patients. The results are presented on FIG. 8

### Example 8

PARP-Inhibition Reverses PAH Development.

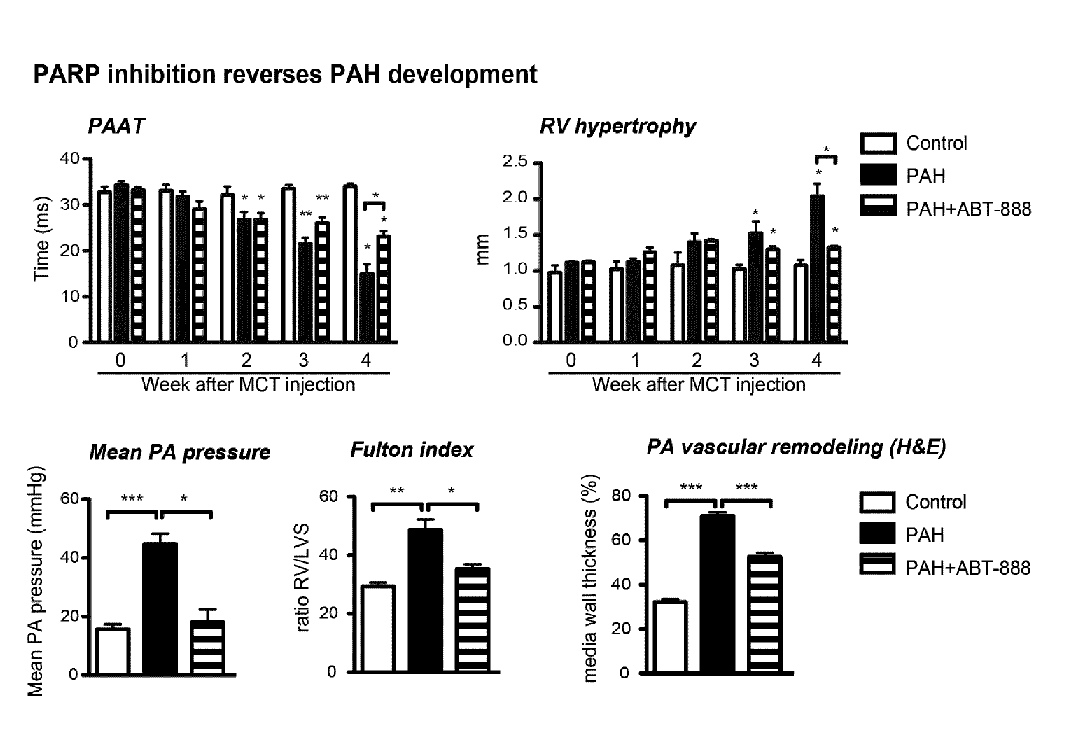

In vivo, ABT-888 (6 mg/kg) was administered per os during 2 weeks after PAH establishment (2 weeks post MCT injection). The effect of ABT-888 was first investigated by a non-invasive echocardiography: we measured a decrease in PAAT (pulmonary artery acceleration time), which is a parameter inversely proportional to the mean PAP, and a decrease in the RV free wall thickness that give information on the state of RV hypertrophy. These results were confirmed invasively by right catherization with a decrease of mean PAP in the rats treated with ABT compared to the rats MCT only. The fulton index showed a decrease in RV Hypertrophy as well and finally, vascular remodeling quantified by H&E staining. Demonstrated that vascular remodeling was nearly normalized. The results are presented on FIG. 9.

### Example 9

PARP-Expression and Activation is Decreased Using ABT-888.

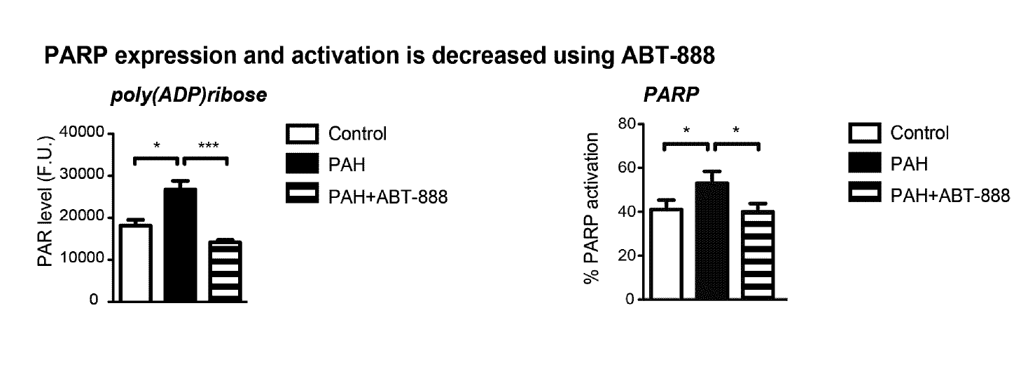

In order to assess the efficiency of our treatment, we quantified PARP and poly(ADP)ribose activities by immunofluorescence on lung histological sections in rats treated or not with ABT-888. As described in (Bonnet et al., 2007a; Bonnet et al., 2009), the poly(ADP)ribose is increased in lung from PAH-rats after 2 weeks of monocrotaline injection, and is significantly decreased by ABT administration. PARP activation is efficiently decreased as well after ABT-888 administration (6mg/kg). The results are presented on FIG. 10.

### Example 10

PAH Model

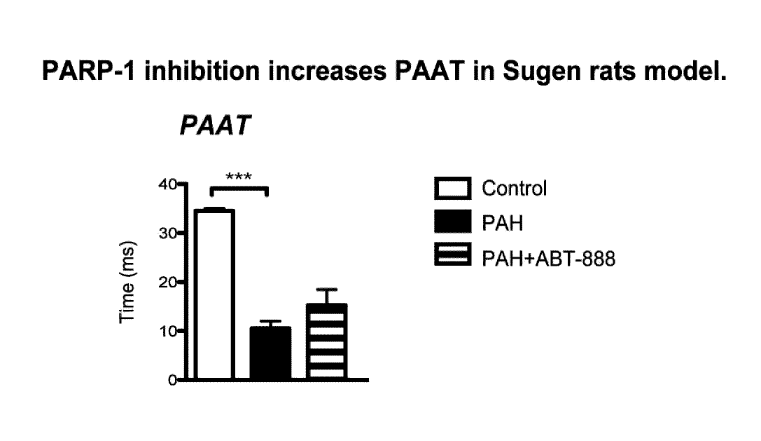

The Sugen rats model is a new PAH experimental model where rats receive SU5416 (s.c.) added with 3 weeks of hypoxia (10% O2) (Abe et al.) After these 3 weeks, the rats return in a normoxic environment for 12 more weeks. This model develop a more sever state of PAH with development of plexiform lesion in the last weeks. After the 3 weeks of hypoxia, PAH development is evaluated by non-invasive techniques (echocardiopgraphy). PAAT is decreased in PAH Sugen rats and reversed with ABT-888 administration (6 mg/kg) at the 7^{th} weeks after SU5416 injection. The results are presented on FIG. 11.

### REFERENCES

Abdallah, Y., Gligorievski, D., Kasseckert, S. A., Dieterich, L., Schafer, M., Kuhlmann, C. R., Noll, T., Sauer, H., Piper, H. M. and Schafer, C. (2007) The role of poly(ADP-ribose) polymerase (PARP) in the autonomous proliferative response of endothelial cells to hypoxia. Cardiovasc Res 73, 568-74.
Abe, K., M. Toba, A. Alzoubi, M. Ito, K. A. Fagan, C. D. Cool, N. F. Voelkel, I. F. McMurtry, and M. Oka. Formation of plexiform lesions in experimental severe pulmonary arterial hypertension. Circulation. 121:2747-54.
Andrabi S A, Kim N S, Yu S W, Wang H, Koh D W, Sasaki M, Klaus J A, Otsuka T, Zhang Z, Koehler R C, Hurn P D, Poirier G G, Dawson V L. Dawson T M. Poly(ADP-ribose) (PAR) polymer is a death signal. Proc Natl Acad Sci USA 2006 Nov. 28; 103(48):18308-13. Epub 2006 Nov. 20.
Archer, S. and Rich, S. (2000) Primary pulmonary hypertension: a vascular biology and translational research “Work in progress”. Circulation 102, 2781-91.
Beller, C. J., Radovits, T., Kosse, J., Gero, D., Szabo, C. and Szabo, G. (2006) Activation of the peroxynitrite-poly(adenosine diphosphate-ribose) polymerase pathway during neointima proliferation: a new target to prevent restenosis after endarterectomy. J Vasc Surg 43, 824-30.
Bonnet. S., Archer, S. L., (2007) Potassium channel diversity in the pulmonary arteries and pulmonary veins: implications for regulation of the pulmonary vasculature in health and during pulmonary hypertension. Pharmacol Ther. July; 115(1):56-69. Epub 2007 Apr. 21.
Bonnet, S., Archer, S. L., Allalunis-Turner, J., Haromy, A., Beaulieu, C., Thompson, R., Lee, C. T., Lopaschuk, G. D., Puttagunta, L., Harry, G., Hashimoto, K., Porter, C. J., Andrade, M. A., Thebaud, B. and Michelakis, E. D. (2007a) A mitochondria-K+ channel axis is suppressed in cancer and its normalization promotes apoptosis and inhibits cancer growth. Cancer Cell 11, 37-51.
Bonnet, S., Michelakis, E. D., Porter, C. J., Andrade-Navarro, M. A., Thebaud, B., Haromy, A., Harry, G., Moudgil, R., McMurtry, M. S., Weir, E. K. and Archer, S. L. (2006) An abnormal mitochondrial-hypoxia inducible factor-1alpha-Kv channel pathway disrupts oxygen sensing and triggers pulmonary arterial hypertension in fawn hooded rats: similarities to human pulmonary arterial hypertension. Circulation 113, 2630-41.
Bonnet, S., Paulin, R., Sutendra, G., Dromparis, P., Roy, M., Watson, K. O., Nagendran, J., Haromy, A., Dyck, J. R. and Michelakis, E. D. (2009) Dehydroepiandrosterone reverses systemic vascular remodeling through the inhibition of the Akt/GSK3-{beta}/NFAT axis. Circulation 120, 1231-40.
Bonnet, S., Rochefort, G., Sutendra, G., Archer, S. L., Haromy, A., Webster, L., Hashimoto, K., Bonnet, S. N. and Michelakis, E. D. (2007b) The nuclear factor of activated T cells in pulmonary arterial hypertension can be therapeutically targeted. Proc Natl Acad Sci USA 104, 11418-23.
Christman, B. W., McPherson, C. D., Newman, J. H., King, G. A., Bernard, G. R., Groves, B. M. and Loyd, J. E. (1992) An imbalance between the excretion of thromboxane and prostacyclin metabolites in pulmonary hypertension. N Engl J Med 327, 70-5.
Dupuis, J. and Hoeper, M. M. (2008) Endothelin receptor antagonists in pulmonary arterial hypertension. Eur Respir J 31, 407-15.
Fong, P. C., Boss, D. S., Yap, T. A., Tutt, A., Wu, P., Mergui-Roelvink, M., Mortimer, P., Swaisland, H., Lau, A., O'Connor, M. J., Ashworth, A., Carmichael, J., Kaye, S. B., Schellens, J. H. and de Bono, J. S. (2009) Inhibition of poly(ADP-ribose) polymerase in tumors from BRCA mutation carriers. N Engl J Med 361, 123-34.
Ghofrani, H. A., Seeger, W. and Grimminger, F. (2005) Imatinib for the treatment of pulmonary arterial hypertension. N Engl J Med 353, 1412-3.
Humbert, M., Morrell, N. W., Archer, S. L., Stenmark, K. R., MacLean, M. R., Lang, I. M., Christman, B. W., Weir, E. K., Eickelberg, O., Voelkel, N. F. and Rabinovitch, M. (2004) Cellular and molecular pathobiology of pulmonary arterial hypertension. J Am Coll Cardiol 43, 13S-24S.
Li, B., Yang, L., Shen, J., Wang, C. and Jiang, Z. (2007) The antiproliferative effect of sildenafil on pulmonary artery smooth muscle cells is mediated via upregulation of mitogen-activated protein kinase phosphatase-1 and degradation of extracellular signal-regulated kinase 1/2 phosphorylation. Anesth Analg 105, 1034-41, table of contents.
McMurtry, M. S., Archer, S. L., Altieri, D. C., Bonnet, S., Haromy, A., Harry, G., Puttagunta, L. and Michelakis, E. D. (2005) Gene therapy targeting survivin selectively induces pulmonary vascular apoptosis and reverses pulmonary arterial hypertension. J Clin Invest 115, 1479-91.
Olabisi, O. A., Soto-Nieves, N., Nieves, E., Yang, T. T., Yang, X., Yu, R. Y., Suk, H. Y., Macian, F. and Chow, C. W. (2008) Regulation of transcription factor NFAT by ADP-ribosylation. Mol Cell Biol 28, 2860-71.
Perros, F., Montani, D., Dorfmuller, P., Durand-Gasselin, I., Tcherakian, C., Le Pavec, J., Mazmanian, M., Fadel, E., Mussot, S., Mercier, O., Herve, P., Emilie, D., Eddahibi, S., Simonneau, G., Souza, R. and Humbert, M. (2008) Platelet-derived growth factor expression and function in idiopathic pulmonary arterial hypertension. Am J Respir Crit Care Med 178, 81-8.
Platoshyn, O., Golovina, V. A., Bailey, C. L., Limsuwan, A., Krick, S., Juhaszova, M., Seiden, J. E., Rubin, L. J. and Yuan, J. X. (2000) Sustained membrane depolarization and pulmonary artery smooth muscle cell proliferation. Am J Physiol Cell Physiol 279, C1540-9.
Steudel, W., Kramer, H. J., Degner, D., Rosseau, S., Schutte, H., Walmrath, D. and Seeger, W. (1997) Endotoxin priming of thromboxane-related vasoconstrictor responses in perfused rabbit lungs. J Appl Physiol 83, 18-24.
Stewart, D. J., Levy, R. D., Cernacek, P. and Langleben, D. (1991) Increased plasma endothelin-1 in pulmonary hypertension: marker or mediator of disease? Ann Intern Med 114, 464-9.
Valdor, R., Schreiber, V., Saenz, L., Martinez, T., Munoz-Suano, A., Dominguez-Villar, M., Ramirez, P., Parrilla, P., Aguado, E., Garcia-Cozar, F. and Yelamos, J. (2008) Regulation of NFAT by poly(ADP-ribose) polymerase activity in T cells. Mol Immunol 45, 1863-71.
Wong, W. K., Knowles, J. A. and Morse, J. H. (2005) Bone morphogenetic protein receptor type II C-terminus interacts with c-Src: implication for a role in pulmonary arterial hypertension. Am J Respir Cell Mol Biol 33, 438-46.
Yuan, X. J. (1995) Voltage-gated K₊currents regulate resting membrane potential and [Ca2+]i in pulmonary arterial myocytes. Circ Res 77, 370-8.

1-9. (canceled)

10. A pharmaceutical composition comprising at least one PARP inhibitor or a pharmaceutically acceptable thereof for the treatment of Group 1 pulmonary arterial hypertension.

11. The pharmaceutical composition of claim 10 further comprising at least one additional compound indicated for the treatment of pulmonary arterial hypertension.

12. The pharmaceutical composition of claim 11 wherein the compound indicated for the treatment of pulmonary arterial hypertension is a calcium channel antagonist, an anticoagulant, endothelin receptor blockers, phosphodiesterease inhibitors, angiotensin-converting enzyme inhibitors or diuretics

13. The pharmaceutical composition of claim 11 wherein the compound indicated for the treatment of pulmonary arterial hypertension is nifedipine, diltiazem, eprostacyclin, prostacyclin, iloprost, flolan treprostinil, adenosine, inhaled nitric oxide, warfarin, digoxin, bosentan, sildenafil, norepinephrine or enalapril

14. The pharmaceutical composition of claim 10 further comprising a pharmaceutically acceptable carrier.

15. A method of treating a subject suffering from Group 1 pulmonary arterial hypertension (PAH), for reducing medial thickness of the pulmonary arteries of a subject suffering from Group 1 pulmonary arterial hypertension, or for inhibiting or reducing Pulmonary Artery Smooth Muscle (PASMC) proliferation and resistance to apoptosis through a NFAT-dependent mechanism which comprises administering to said subject in need of such treatment a dose effective against PAH of at least one PARP inhibitor or a pharmaceutically acceptable salt thereof.

16. The method of claim 15 wherein the Group 1 PAH is:
a. idiopathic or primary pulmonary hypertension,
b. familial hypertension,
c. pulmonary hypertension secondary to, but not limited to, connective tissue disease, congenital heart defects (shunts), pulmonary fibrosis, portal hypertension, HIV infection, sickle cell disease, drugs and toxins (e.g., anorexigens, cocaine), chronic hypoxia, chronic pulmonary obstructive disease, sleep apnea, and schistosomiasis,
d. pulmonary hypertension associated with significant venous or capillary involvement (pulmonary veno-occlusive disease, pulmonary capillary hemangiomatosis),
e. secondary pulmonary hypertension that is out of proportion to the degree of left ventricular dysfunction, or
f. persistent pulmonary hypertension in newborn babies, which comprises administering to said human in need of such treatment a dose effective against the respective disorder of at least one PARP inhibitor or a pharmaceutically acceptable salt thereof.

17. The method according to claim 15 wherein the subject is human.

18. The method according to claim 15 wherein the PARP inhibitor from the class of Nicotinamides, Benzamides, Isoquinolinones, Dihydroisoquinolinones, Benzimidazoles, indoles, Phthalazin-1 (2H)-ones, quinazolinones, Isoindolinones, Phenanthridines, phenanthhdinones, Benzopyrones, Unsaturated hydroximic acid derivatives or Pyridazines.

19. The method according to claim 15 wherein the PARP inhibitor is NU1025, ABT-888(Veliparib), Olaparib (was AZD-2281), CEP 9722, MK4827, AG014699, Iniparib (previously BSI 201), LT-673, 3-aminobenzamide or E7016.

20. The method according to claim 19 wherein PARP inhibitor is ABT-888.

21. The method according to claim 15 further comprising administering at least one additional compound indicated for the treatment of pulmonary arterial hypertension.

22. The method of claim 21 wherein the compound indicated for the treatment of pulmonary arterial hypertension is a calcium channel antagonist, an anticoagulant, endothelin receptor blockers, phosphodiesterease inhibitors, angiotensin-converting enzyme inhibitors or diuretics.

23. The method of claim 21 wherein the compound indicated for the treatment of pulmonary arterial hypertension is nifedipine, diltiazem, eprostacyclin, prostacyclin, iloprost, flolan treprostinil, adenosine, inhaled nitric oxide, warfarin, digoxin, bosentan, sildenafil, norepinephrine or enalapril.

24. A method of diagnosing group 1 pulmonary arterial hypertension (PAH) in a subject comprising determining the PARP level in a biological sample of the subject wherein an elevated PARP level compared to a reference sample indicates that the subject suffers from group 1 PAH.

25. A method of diagnosing group 1 pulmonary arterial hypertension (PAH) in a subject comprising determining the PARP regulation in a biological sample of the subject wherein an up-regulated PARP level compared to a reference sample indicates that the subject suffers from group 1 PAH.

26. A method for evaluating the likelihood of group 1 pulmonary arterial hypertension (PAH) in a subject comprising:
1. comparing a PARP level in a biological sample from a subject to be tested to a reference PARP level obtained from a healthy subject; and
2. determining if the level of PARP in said biological sample is different from the level of the reference PARP;
wherein determination of a difference is indicative of the likelihood of group 1 PAH in said subject to be tested.

27. The method of claim 26 wherein the level of PARP in said biological sample is elevated compared to the reference PARP.

28. The method of claim 24 wherein the patient is human.

29. The method of claim 25 wherein the reference is the level present in subject not suffering group 1 PAH.

30. The method according to claim 25 wherein the biological sample is lung or blood.
